(19) United States
(12) Patent Application Publication
(10) Pub. No.: US 2006/0051374 A1
(43) Pub. Date: Mar. 9, 2006

(54) Compositions and methods for mucosal vaccination

(75) Inventors: Richard L. Miller, Maplewood, MN (US); William C. Kieper, Stillwater, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/116,476

(22) Filed: Apr. 28, 2005

Related U.S. Application Data
(60) Provisional application No. 60/566,121, filed on Apr. 28, 2004.

(51) Int. Cl. A61K 39/12; A61K 39/02; A61K 31/4745

(52) U.S. Cl. 424/204.1; 424/234.1; 514/291

(57) ABSTRACT

The present invention provides pharmaceutical combinations that include an IRM compound formulated for mucosal administration and an antigen formulated for mucosal administration. Additionally, the invention provides methods for immunizing a subject. Generally, the methods include administering an antigen to a mucosal surface of the subject in an amount effective, in combination with an IRM compound, to generate an immune response against the antigen; and administering an IRM compound to a mucosal surface of the subject in an amount effective, in combination with the antigen, to generate an immune response against the antigen.

### CROSS-REFERENCE TO RELATED APPLICATIONS

This application claims priority to U.S. Provisional Patent Application Ser. No. 60/566,121, filed April 28, 2004.

### BACKGROUND

Classical injection vaccination routes—e.g., subcutaneous, intramuscular, and intravenous—are primarily concerned with the induction of systemic immunity (blood serum antibodies and T cells). While this approach may be appropriate against diseases caused by infectious agents which gain systemic access to the body through punctured or damaged skin (e.g. tetanus), most pathogens naturally infect hosts through mucosal routes such as, for example, oral, nasal, or urogenital mucosa.

Injectable vaccines are generally ineffective for eliciting immunity at mucosal surfaces, which is typically mediated through the production and secretion of IgA and secreted IgA (s-IgA), which is secreted into the lumen of the intestinal, respiratory, and urinary tract, often with the secretion products of various glandular tissues. In these secretions, s-IgA is able to bind to the pathogen, which allows immune cells to eliminate the pathogen before the pathogen can begin to infect cells of the host. Thus, mucosal vaccination can substantially reduce the likelihood of a pathogen infecting host cells (i.e., cellular infection) and, in some cases, even prevent a pathogen from infecting host cells. In contrast, injected vaccines often respond to antigens released as a result of host cell infection by the pathogen (e.g., lysis of infected cells). Thus, one important distinction between mucosal vaccination and injected vaccination is that mucosal vaccination can stimulate a host's defenses to limit or even prevent cellular infection, whereas injected vaccination responds to a consequence of cellular infection, hopefully before the infectious disease develops.

Mucosal vaccines are likely to be more effective at preventing or limiting mucosal infections due to their ability to induce an s-IgA response. In addition, mucosal vaccines offer several other advantages over injectable vaccines. These advantages include easier administration, reduced side effects, administration is non-invasive (e.g., does not require needles), and the potential for almost unlimited frequency of boosting without the need for trained personnel. These advantages can reduce the cost and increase the safety of vaccinations and improve compliance, issues especially important in the developing world. Furthermore, improvements in the design of novel mucosal vaccination systems may allow the development of vaccines against diseases that are currently poorly controlled.

Additionally, induction of a mucosal immune response at one mucosal site may result in an immune response at a distant mucosal site. For example, nasal or oral mucosal vaccination can generate secretion of s-IgA and IgG from the vaginal mucosa.

Despite the important advantages of immunizing through mucosal routes, success with mucosal immunizations has been limited due to many factors including, for example, degradation of antigens, limited adsorption and interaction with nonspecific host factors at mucosal sites, a lack of safe and effective adjuvants, and the use of inadequate delivery systems. There is a substantial ongoing need to expand the utility and efficacy of mucosal vaccines.

### SUMMARY

It has been found that certain small molecule immune response modifiers (IRMs) can be useful as components of pharmaceutical combinations suitable for mucosal delivery.

Accordingly, the present invention provides a pharmaceutical combination that includes an IRM compound formulated for mucosal administration, and an antigen formulated for mucosal administration. In some embodiments, the IRM compound and the antigen may be provided in a single formulation, while in other embodiments, the IRM compound and antigen may be provided in separate formulations.

In another aspect, the present invention also provides a method of immunizing a subject. Generally, the method includes administering an antigen to a mucosal surface of the subject in an amount effective, in combination with an IRM compound, to generate an immune response against the antigen; and administering an IRM compound to a mucosal surface of the subject in an amount effective, in combination with the antigen, to generate an immune response against the antigen.

In some embodiments, the method may further include one or more priming doses of antigen, one or more booster doses of antigen or IRM compound, or both.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is flow cytometry data showing proliferation of antigen-specific T cells in lymphatic tissues (NALT, FIG. 1A; ILN, FIG. 1B; CLN, FIG. 1C; spleen, FIG. 1D) after vaccination.

FIG. 2 is data showing the total number of antigen-specific T cells in lymphoid tissues (FIG. 2A-2C) and showing the percentage of antigen-specific T cells in the nasal mucosa (FIG. 2D) after vaccination.

FIG. 3 is flow cytometry data demonstrating the expansion of antigen-specific CD8⁺ T cells (FIG. 3A) and CD4⁺ T cells (FIG. 3B) after vaccination.

FIG. 4 is data showing lung lavage IgA (FIG. 4A), nasal lavage IgA (FIG. 4B), and serum IgG (FIG. 4C) antibody responses to immunization via various routes with a combination of IRM and antigen.

FIG. 5 is data showing lung lavage IgA (FIG. 5A) and serum IgG2b (FIG. 5B) antibody responses to intranasal administration of antigen alone or with various IRM compounds.

FIG. 6 is data showing the of antigen-specific T cells in the DLN (FIG. 6A) and spleen (FIG. 6B) after immunization with antigen and one of various IRM compounds.

FIG. 7 is data showing the of antigen-specific T cells in the DLN (FIG. 7A), and NALT (FIG. 7B) when immunized twice five months apart through various routes with antigen and IRM compound.

### DETAILED DESCRIPTION OF ILLUSTRATIVE EMBODIMENTS OF THE INVENTION

Immune response modifiers (IRMs) are compounds that can possess potent immunomodulating activity. IRMs appear to act through basic immune system mechanisms known as Toll-like receptors (TLRs) to selectively modulate cytokine biosynthesis. For example, certain IRM compounds induce the production and secretion of certain cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-I and TNF (U.S. Pat. No. 6,518,265). Certain IRMs may be useful for treating a wide variety of diseases and conditions such as, for example, certain viral diseases (e.g., human papilloma virus, hepatitis, herpes), certain neoplasias (e.g., basal cell carcinoma, squamous cell carcinoma, actinic keratosis, melanoma), and certain T_{H}2-mediated diseases (e.g., asthma, allergic rhinitis, atopic dermatitis).

The present invention relates to pharmaceutical combinations that can be effective for use as mucosal vaccines and methods that include administering such a combination to a mucosal surface. Generally, a pharmaceutical composition according to the invention includes an IRM compound and an antigen, each formulated in a manner suitable for mucosal delivery and each in an amount that, in combination with the other, can raise an immune response against the antigen. The benefits of mucosal vaccination are many; the compositions and methods of the invention may provide one or more of the following:

1) The composition may be easily administered without the need for needles;

2) Mucosal vaccination can generate both a mucosal and a systemic immune response, whereas injected vaccines generally induce only a systemic response. Because most pathogens infect a host at a mucosal surface, mucosal vaccination induces an immune response at the site of pathogen entry; and

3) Mucosal vaccination can induce an immune response at a mucosal site other than the vaccination site.

Components of such a pharmaceutical combination may be said to be delivered “in combination” with one another if the components are provided in any manner that permits the biological effect of contacting one component with cells to be sustained at least until another component is contacted with the cells. Thus, components may be delivered in combination with one another even if they are provided in separate formulations, delivered via different routes of administration, and/or administered at different times.

For example, an IRM compound and an antigen may be considered a pharmaceutical combination regardless of whether the components are provided in a single formulation or the antigen is administered in one formulation and the IRM compound is administered in a second formulation. When administered in different formulations, the components may be administered at different times, if desired, but administered so that the immune response generated is greater than the immune response generated if either the antigen or the IRM compound is administered alone.

In some embodiments, the pharmaceutical combination may include an IRM/antigen conjugate in which at least one IRM moiety is covalently attached to an antigen. Methods of preparing such IRM/antigen conjugates are described, for example, in U.S. patent Publication No. 2004/0091491.

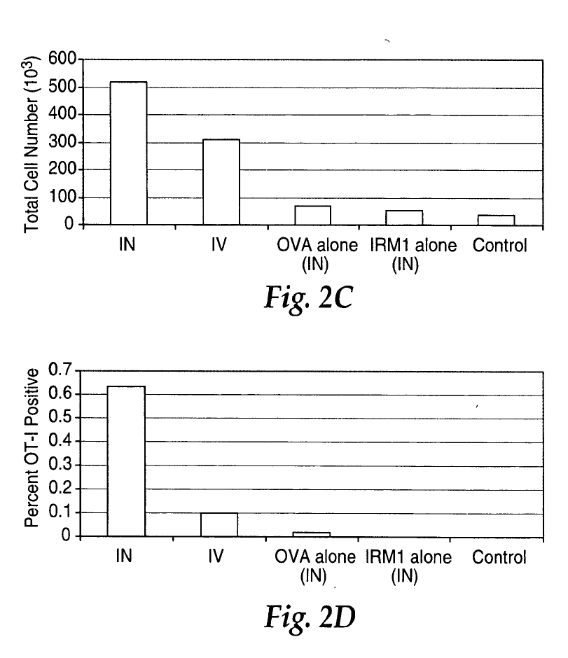

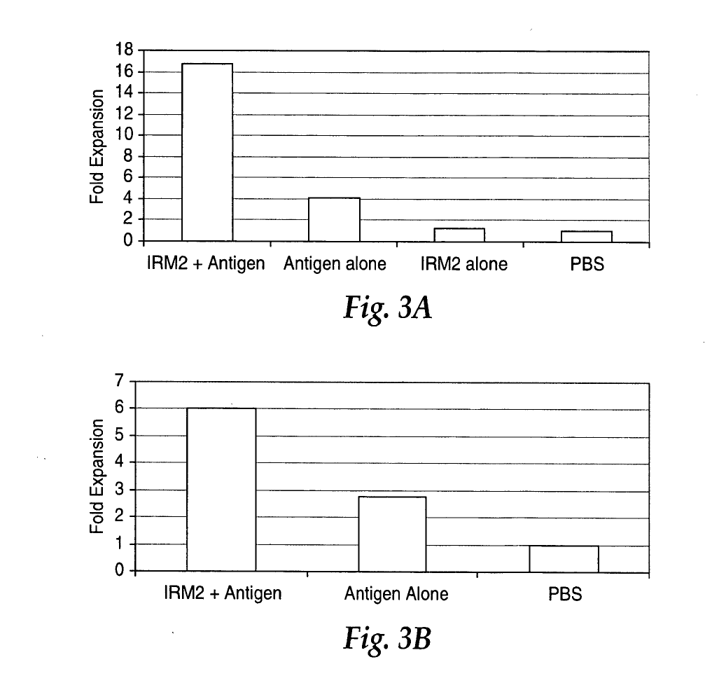

One method of measuring an immune response induced by a mucosal vaccine is to measure the expansion of antigen-specific CD8⁺ T cells in response to challenge with the antigen. This is shown in Example 1. Antigen-specific CD8⁺ T cells were fluorescently labeled and adoptively transferred into syngenic mice. The mice were challenged with an IRM-antigen conjugate. Four days later, lymphoid tissue from various sites (nasal associated lymphatic tissue (NALT), cervical lymph node (CL), and spleen) was removed and the expansion of antigen-specific CD8⁺ T cells was measured. In the tissue from each site, expansion of CD8⁺ T cells was greater as a result of intranasal immunization than as a result of intravenous immunization with the IRM-antigen conjugate, or intranasal immunization with either antigen or IRM (FIG. 2A-2C). Likewise, a greater percentage of antigen-specific CD8⁺ T cells were observed in the nasal mucosa seven days after intranasal immunization with the IRM-antigen conjugate than were observed following either intravenous immunization with the IRM-antigen conjugate, or intranasal immunization with either antigen or IRM (FIG. 2D). Similar results were found using non-conjugated IRM and antigen (FIG. 3A).

Another method of measuring an immune response induced by mucosal vaccination is to measure expansion of antigen-specific CD4⁺ T cells in lymphoid tissue such as, for example, nasal associated lymphoid tissue. Activated antigen-specific CD4⁺ T cells, in turn, stimulate B cells to produce antibodies (e.g., s-IgA) directed against the antigen. In Example 2, antigen-specific T cells were adoptively transferred into host mice. The mice were challenged with a combination of IRM compound and an immunogenic antigen peptide. Three days later, lymphoid tissue was removed from the mice and expansion of antigen-specific CD4⁺ T cells was analyzed. Results are shown in FIG. 3B. Expansion of CD4⁺ T cells was greater in mice immunized with IRM and antigen than in mice immunized with antigen alone.

Thus, a mucosal route of vaccination (e.g., intranasal) can provide a greater number of antigen-specific CD8⁺ T cells and/or CD4⁺ T cells at relevant tissue sites—the nasal associated lymphoid tissue (NALT) and the nasal mucosa—compared to either non-mucosal route of delivery (intravenous), or mucosal delivery of either antigen alone or IRM alone. Expansion of the antigen-specific T cell population at mucosal sites indicates activation of immune cells in those locations and the generation of an immune response that can protect against infection. When both antigen-specific CD8⁺ T cells and antigen-specific CD4⁺ T cells are activated, both an antigen-specific cell-mediated immune response and an antigen-specific antibody immune response may be generated.

The antigen can include any material that raises a mucosal immune response. Suitable antigenic materials include but are not limited to proteins; peptides; polypeptides; lipids; glycolipids; polysaccharides; carbohydrates; polynucleotides; prions; live or inactivated bacteria, viruses or fungi; and bacterial, viral, fungal, protozoal, tumor-derived, or organism-derived immunogens, toxins or toxoids. Additionally, as used herein, an antigen may include an oligonucleotide sequence that does not necessarily raise a mucosal immune response itself, but can be expressed in cells of the host to produce an antigenic protein, peptide, or polypeptide. Such oligonucleotides are useful, for example, in DNA vaccines. In some embodiments, the antigen may include a combination of two or more antigenic materials.

Conditions for which a composition that includes an IRM and an antigen, each formulated for mucosal administration may be useful include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) T_{H}2-mediated, atopic diseases, such as atopic dermatitis or eczema, eosinophilia, asthma, allergy, allergic rhinitis, and Ommen's syndrome.

For example, a mucosally administered composition may be used for prophylactic or therapeutic protection against, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, and Alzheimer's Disease.

In some cases, mucosal vaccination may be useful for decreasing the likelihood of, or even preventing, infection across a mucosal surface. In other cases, a mucosal vaccine may be useful for stimulating a serum antibody response. In some cases, a mucosal vaccine may provide both protection against mucosal infection and a serum antibody response. Thus, mucosal vaccination may be useful for vaccination against pathogens that do not typically infect across a mucosal surface.

In some embodiments, the antigen may be administered in one or more separate “priming” doses prior to administration of the antigen-IRM combination. Priming in this way may provide an increased immune response upon administration of the antigen-IRM combination.

In other embodiments, the antigen may be administered in one or more separate “booster” doses after administration of the antigen-IRM combination. Boosting in this way may reinvigorate an at least partially resolved immune response by activating CD8⁺ memory T cells, CD4⁺ memory T cells, or both.

In still other embodiments, an IRM compound may be administered in one or more separate booster doses after administration of the antigen-IRM combination. The IRM compound provided in a booster dose may be the same or different that the IRM compound provided in the antigen-IRM combination, and may be the same or different than the IRM compound provided in any other booster dose. Moreover, any combination of IRM compounds may be used, whether as the IRM component of an antigen-IRM combination or as a booster.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929) and more are still being discovered. Other IRMs have higher molecular weights, such as oligonucleotides, including CpGs (see, e.g., U.S. Pat. No. 6,194,388).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. patent Publication Nos. 2004/0091491; 2004/0147543; and 2004/0176367; and International Publication Nos. WO 2005/18551, WO 2005/18556, and WO 2005/20999.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. 6,387,938), certain derivatives of a 4-aminopyrimidine fused to a five membered nitrogen containing heterocyclic ring (such as adenine derivatives described in U. S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

In certain embodiments, the IRM compound may be an amide substituted imidazoquinolin amine such as, for example, 1-(2-amino-2-methylpropyl)-2-(ethoxymethyl)-1H-imidazo[4,5-c]quinolin-4-amine or N-[6-( {2-[4-amino-2-(ethoxymethyl)-1H-imidazo[4,5-c]quinolin-1-yl]-1,1-dimethylethyl}amino)-6-oxohexyl]-4-azido-2-hydroxybenzamide.

In other embodiments, the IRM compound may be a thiazoloquinoline amine such as, for example, 2-butylthiazolo[4,5-c]quinolin-4-amine.

In other embodiments, the IRM compound may be an imidazoquinoline amine such as, for example, 4-amino-α,α-dimethyl-2-ethoxymethyl-1H-imidazo[4,5-c]quinolin-1-ethanol.

In other embodiments, the IRM compound may be an amide substituted imidazoquinoline amine such as, for example, N-{3-[4-amino-1-(2-methylpropyl)-1H-imidazo[4,5-c]quinolin-7-yloxy]propyl}nicotinamide.

In other embodiments, the IRM compound may be a sulfonamide substituted imidazoquinoline amine such as, for example, 3-[4-amino-2-(ethoxymethyl)-1H-imidazo[4,5-c]quinolin-1-yl]-N,2,2-trimethylpropane-1-sulfonamide.

In other embodiments, the IRM compound may be a thioether substituted imidazoquinoline amine such as, for example, 2-butyl-1-{2-methyl-2-[2-(methylsulfonyl)ethoxy]propyl}-1H-imidazo[4,5-c]quinolin-4-amine.

In other embodiments, the IRM compound may be a pyrazoloquinoline amine such as, for example, 2-butyl-1-[2-(propylsulfonyl)ethyl]-2H-pyrazolo[3,4-c]quinolin-4-amine.

In other embodiments, the IRM compound may be an arylalkyleneoxy substituted imidazoquinoline amine such as, for example, 1-{4-amino-2-ethoxymethyl-7-[3-(pyridin-3-yl)propoxy]-1H-imidazo[4,5-c]quinolin-1-yl}-2-methylpropan-2-ol.

In other embodiments, the IRM compound may be a urea substituted imidazopyridine amine such as, for example, N-{2-[4-amino-2-(ethoxymethyl)-6,7-dimethyl-1H-imidazo[4,5-c]pyridin-1-yl]-1,1-dimethylethyl}-N′-cyclohexylurea.

In other embodiments, the IRM compound may be a sulfonamide substituted imidazoquinoline amine such as, for example, N-[2-(4-amino-2-butyl-1H-imidazo[4,5-c]quinolin-1-yl)-1,1-dimethylethyl]methanesulfonamide.

In still other embodiments, the IRM compound may be an amide substituted imidazoquinoline amine such as, for example, N-{2-[4-amino-2-(ethoxymethyl)-1H-imidazo[4,5-c]quinolin-1-yl]-1,1-dimethylethyl}cyclohexanecarboxamide.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound may be an agonist of at least one TLR, preferably an agonist of TLR6, TLR7, or TLR8. In certain embodiments, the IRM compound may be a TLR8-selective agonist. In other embodiments, the IRM compound may be a TLR7-selective agonist. As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8.

A TLR8-selective agonist or a TLR7-selective agonist may act as an agonist for the indicated TLR and one or more of TLR1, TLR2, TLR3, TLR4, TLR5, TLR6, TLR9, or TLR10. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6.

The TLR agonism for a particular compound may be assessed in any suitable manner. For example, assays for detecting TLR agonism of test compounds are described, for example, in U.S. patent Publication No. US2004/0132079, and recombinant cell lines suitable for use in such assays are described, for example, in International Patent Publication No. WO 04/053057.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

A component of an antigen-IRM combination, as well as an antigen or IRM provided in a priming dose or booster dose, may be provided in any formulation suitable for mucosal administration to a subject. Suitable types of formulations are described, for example, in U.S. Pat. No. 5,939,090; U.S. Pat. No. 6,365,166; U.S. Pat. No. 6,245,776; and U.S. Pat. No. 6,486,168. The compound—whether antigen or IRM compound—may be provided in any suitable form including but not limited to a solution, a suspension, an emulsion, or any form of mixture. The compound may be delivered in formulation with any pharmaceutically acceptable excipient, carrier, or vehicle. Moreover, the IRM component and antigen component of an antigen-IRM combination may be provided together in a single formulation or may be provided in separate formulations. A formulation may be delivered in any suitable dosage form such as, for example, a cream, an ointment, an aerosol formulation, a non-aerosol spray, a gel, a lotion, and the like. The formulation may further include one or more additives including but not limited to adjuvants, penetration enhancers, colorants, fragrances, flavorings, moisturizers, thickeners, and the like.

A formulation may be administered to any suitable mucosal surface of a subject such as, for example, oral, nasal, or urogenital mucosa.

The composition of a formulation suitable for mucosal vaccination will vary according to factors known in the art including but not limited to the physical and chemical nature of the component(s) (i.e., the IRM compound and/or antigen), the nature of the carrier, the intended dosing regimen, the state of the subject's immune system (e.g., suppressed, compromised, stimulated), the method of administering the component(s), and the species to which the formulation is being administered. Accordingly, it is not practical to set forth generally the composition of a formulation effective for mucosal vaccination for all possible applications. Those of ordinary skill in the art, however, can readily determine an appropriate formulation with due consideration of such factors.

In some embodiments, the methods of the present invention include administering IRM to a subject in a formulation of, for example, from about 0.0001% to about 10% (unless otherwise indicated, all percentages provided herein are weight/weight with respect to the total formulation) to the subject, although in some embodiments the IRM compound may be administered using a formulation that provides IRM compound in a concentration outside of this range. In certain embodiments, the method includes administering to a subject a formulation that includes at least about 0.01% IRM compound, at least about 0.03% IRM compound, or at least about 0.1% IRM compound. In other embodiments, the method includes administering to a subject a formulation that includes up to about 5% IRM compound, up to about 1% IRM compound, or up to about 0.5% IRM compound. In one particular embodiment, the method includes administering the IRM compound in a formulation that includes from at least about 0.1% IRM compound up to about 5% IRM compound.

In some embodiments, a formulation may be administered to the mucosal surface that is a typical or expected site of infection by a particular pathogen. For example, a mucosal vaccine, or a component of a mucosal vaccine may be administered to the nasal mucosa in order to vaccinate against a respiratory pathogen (e.g., an influenza virus). Alternatively, a formulation may be administered to one mucosal surface in order to induce an immune response at a distant mucosal site. For example, a formulation may be administered to the nasal mucosa or oral mucosa in order to vaccinate against a pathogen that can infect through, for example, the vaginal mucosa (e.g., a herpesvirus).

An amount of an IRM compound effective for mucosal vaccination is an amount sufficient to increase an immune response to the antigen in the combination compared to the immune response raised by administering the antigen without the IRM compound. The precise amount of IRM compound administered in a mucosal vaccine will vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the species to which the mucosal vaccine is being administered. Accordingly, it is not practical to set forth generally the amount that constitutes an amount of IRM compound effective for mucosal vaccination for all possible applications. Those of ordinary skill in the art, however, can readily determine the appropriate amount with due consideration of such factors.

In some embodiments, the methods of the present invention include administering sufficient IRM compound to provide a dose of, for example, from about 100 ng/kg to about 50 mg/kg to the subject, although in some embodiments the methods may be performed by administering IRM compound in a dose outside this range. In some of these embodiments, the method includes administering sufficient IRM compound to provide a dose of from about 10 μg/kg to about 5 mg/kg to the subject, for example, a dose of about 3.75 mg/kg.

The dosing regimen may depend at least in part on many factors known in the art including but not limited to the physical and chemical nature of the IRM compound, the nature of the carrier, the amount of IRM being administered, the state of the subject's immune system (e.g., suppressed, compromised, stimulated), the method of administering the IRM compound, and the species to which the mucosal vaccine is being administered. Accordingly it is not practical to set forth generally the dosing regimen effective for mucosal vaccination for all possible applications. Those of ordinary skill in the art, however, can readily determine an appropriate dosing regimen with due consideration of such factors.

In some embodiments, the IRM compound may be administered, for example, from once to multiple doses within a set time period (e.g., daily, per week, etc.). In certain embodiments, the IRM compound may be administered a single time. In other embodiments, the IRM may be administered from about once every ten years to multiple times per day. For example, the IRM compound may be administered at least once every ten years, at least once every five years, or at least once every two years. In other embodiments, the IRM compound may be administered, for example, at least once per year, at least once every six months, at least once per month, at least once per week, or at least once per day. In one particular embodiment, the IRM compound is administered from about once per month to about once per year.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are shown in Table 1.

TABLE 1

| Com- | | |
|---|---|---|
| pound | Chemical Name | Reference |
| IRM1 | N-[6-({2-[4-amino-2-(ethoxymethyl)- | U.S. Pat. No. |
| | 1H-imidazo[4,5-c]quinolin-1-yl]- | 2004/0091491 |
| | 1,1-dimethylethyl}amino)-6-oxohexyl]- | IRM1 |
| | 4-azido-2-hydroxybenzamide | |
| IRM2 | 1-(2-amino-2-methylpropyl)-2- | U.S. Pat. No. |
| | (ethoxymethyl)-1H-imidazo[4,5- | 6,677,349 |
| | c]quinolin-4-amine | Example 164, |
| | | Part I |
| IRM3 | 2-butylthiazolo[4,5-c]quinolin- | U.S. Pat. No. |
| | 4-amine | 6,110,929 |
| | | Example 18 |
| IRM4 | 4-amino-α,α-dimethyl-2-ethoxymethyl- | U.S. Pat. No. |
| | 1H-imidazo[4,5-c]quinolin-1-ethanol | 5,389,640 |
| | | Example 99 |
| IRM5 | N-{3-[4-amino-1-(2-methylpropyl)- | U.S. Ser. No. |
| | 1H-imidazo[4,5-c]quinolin-7- | 60/508634 |
| | yloxy]propyl}nicotinamide | Example 16 |
| IRM6 | 3-[4-amino-2-(ethoxymethyl)-1H- | PCT App. No. |
| | imidazo[4,5-c]quinolin-1-yl]- | US04/43447 |
| | N,2,2-trimethylpropane-1-sulfonamide | Example 36 |
| IRM7 | 2-butyl-1-(2-methyl-2-[2- | PCT App. No. |
| | (methylsulfonyl)ethoxy]propyl}- | US04/40383 |
| | 1H-imidazo[4,5-c]quinolin-4-amine | Example 32 |
| IRM8 | 2-butyl-1-[2-(propylsulfonyl)ethyl]- | PCT App. No. |
| | 2H-pyrazolo[3,4-c]quinolin-4-amine | US04/32480 |
| | | Example 60 |
| IRM9 | 1-{4-amino-2-ethoxymethyl-7-[3- | WO 2005/20999 |
| | (pyridin-3-yl)propoxy]-1H-imidazo[4,5- | Example 122 |
| | c] quinolin-1-yl}-2-methylpropan-2-ol | |
| IRM10 | N-{2-[4-amino-2-(ethoxymethyl)-6,7- | U.S. Pat. No. |
| | dimethyl-1H-imidazo[4,5-c]pyridin-1- | 6,545,017^{#} |
| | yl]-1,1-dimethylethyl}-N′- | |
| | cyclohexylurea | |
| IRM11 | N-[2-(4-amino-2-butyl-1H-imidazo[4,5- | U.S. Pat. No. |
| | c]quinolin-1-yl)-1,1- | 6,677,349^{#} |
| | dimethylethyl]methanesulfonamide | |
| IRM12 | N-{2-[4-amino-2-(ethoxymethyl)-1H- | U.S. Pat. No. |
| | imidazo[4,5-c]quinolin-1-yl]-1,1- | 6,756,382^{#} |
| | dimethylethyl}cyclohexanecarboxamide | |

^{#}This compound is not specifically exemplified but can be readily prepared using the synthetic methods disclosed in the cited reference.

### Example 1

An Ovalbumin-IRM1 conjugate was prepared as follows. IRM1 was suspended in dimethyl sulfoxide (DMSO) to 10 mg/ml. Ovalbumin was suspended in phosphate buffered saline (PBS) to 10 mg/ml and the pH adjusted to >10.0 by the addition of NaOH. 500 μL of the ovalbumin solution (5 mg ovalbumin) was mixed with 100 μL of the IRM1 solution (1 mg IRM1) in a single well of a 12-well tissue culture plate. The plate was placed on ice and a long wavelength UV light source was placed directly over the plate as close to the well containing the IRM1/ovalbumin mixture as possible. The mixture was irradiated for 15 minutes. The resulting conjugate was removed from the well and resuspended in PBS to a final concentration of 5 mg/mL ovalbumin, 0.5 mg/mL IRM1, and dialyzed against PBS to remove any unconjugated IRM.

Chicken Ovalbumin-specific CD8⁺ T cells (OT-I, The Jackson Laboratories, Bar Harbor, Me.) were labeled with carboxyfluoroscein succinimidyl ester (CFSE, Molecular Probes, Inc., Eugene, Oreg.), a fluorescent dye that stains cells in a stabile manner, and then adoptively transferred into syngenic C57BL/6 mice (Charles River Laboratories, Wilmington, Mass.). The recipient mice were then immunized on Day 0 with 100 micrograms (μg) of the Ovalbumin-IRM1 conjugate, either intranasally (IN) or intravenously (IV). On Day 4, the mice were sacrificed and the nasal associated lymphoid tissue (NALT), inguinal lymph nodes (ILN), cervical lymph nodes (CLN), and spleens (Spl) were removed. Each tissue harvested from the mice was run through a 100 μm nylon screen (BD Biosciences, Bedford, Mass.), centrifuged, and resuspended in Flow Cytometry Staining Buffer (Biosource International, Inc., Rockville, Md.). Cells were then labeled with CD8-cychrome (BD Pharmigen, San Diego, Calif.) and SIINFEKL/K^{b} tetramer-phycoerytherine (Beckman Coulter, Inc., Fullerton, Calif.) antibodies. Cells were then run on a FACSCaliber (Becton, Dickinson, and Co., San Jose, Calif.) and CD8⁺SIINFEKL/K^{b} tetramer⁺ T cells were analyzed for CFSE expression.

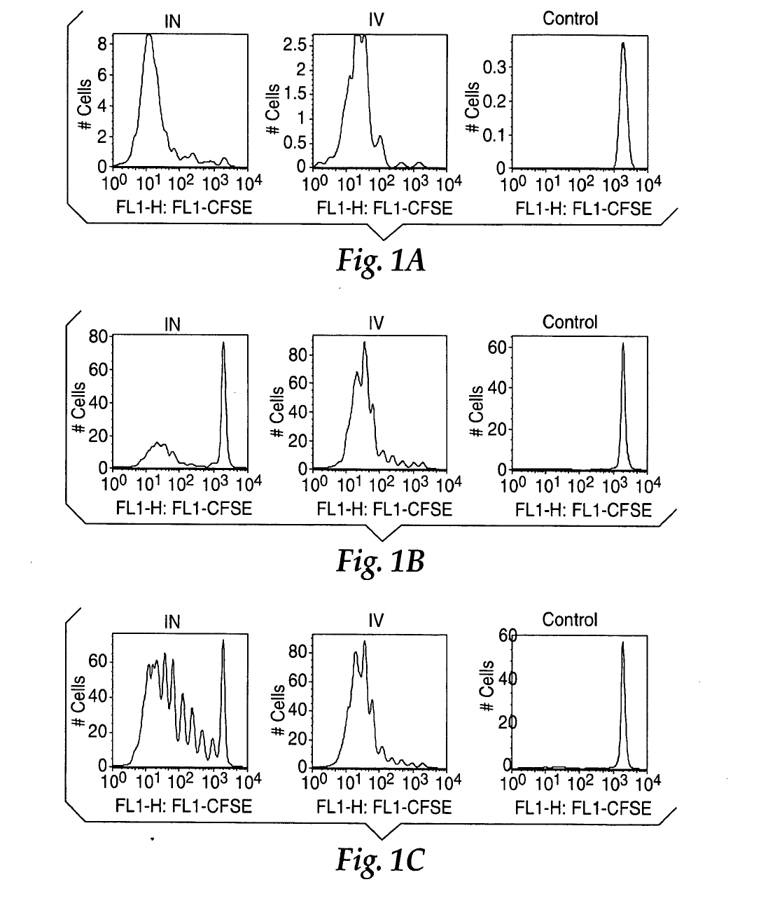

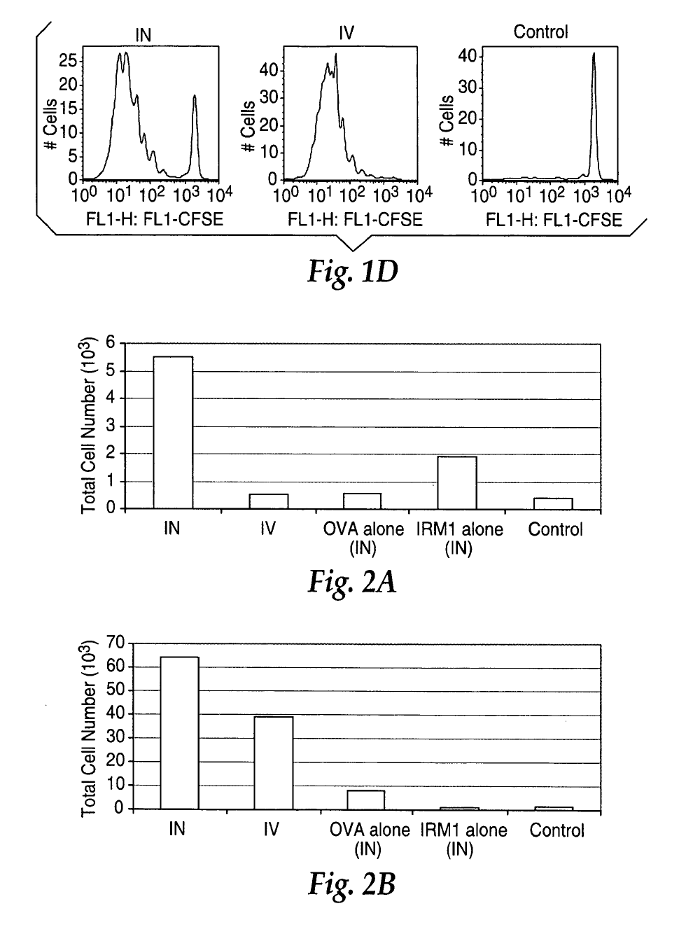

Results are shown in FIG. 1 as follows: NALT in FIG. 1A; ILN in Figure 1B; CLN in FIG. 1C; and Spleen in FIG. 1D. Intranasal delivery of antigen with IRM results in the effective activation of cytotoxic T lymphocytes in all locations, as indicated by a progressive loss of CFSE.

Separately, total OT-I cell numbers at Day 7 were counted in nasal associated lymphoid tissue (NALT), cervical lymph node (CLN), and spleen (Spl). OT-I cell numbers were determined by counting total lymphocytes (Trypan blue exclusion) and multiplying by the percentage of OT-I⁺CD8⁺ (flow cytometry analysis). Additionally, the percentage of OT-I cells in the nasal mucosa at determined at Day 7. Results are shown in FIG. 2 as follows: NALT in FIG. 2A; CLN in FIG. 2B; Spleen in FIG. 2C; and nasal mucosa in FIG. 2D.

Intranasal delivery of antigen plus IRM1 generated greater total OT-I cell numbers at Day 7 than intravenous delivery in all lymphoid tissues examined. Intranasal delivery of IRM1 plus antigen also generated greater total OT-I cell numbers at Day 7 than antigen alone, indicating a dramatic effect of the IRM in enhancing antigen specific T cell activation via that route. Furthermore, the intranasal route of vaccination results in a greater number of OT-I cells at relevant tissue sites—the nasal associated lymphoid tissue (NALT) and the nasal mucosa.

### Example 2

CD8⁺ T cells from OT-I mice (The Jackson Laboratories, Bar Harbor, Me.) were adoptively transferred into C57BL/6 (Charles River Laboratories, Wilmington, Mass.) mice. CD4⁺ T cells from DO.11 TCR mice (The Jackson Laboratories, Bar Harbor, Me.) were adoptively transferred into Balb/c mice (Charles River Laboratories, Wilmington, Mass.). The mice were then immunized intranasally at Day 0 as follows: OT-I-transferred C57BL/6 mice were immunized with 100 μg whole chicken ovalbumin per mouse, either with (IRM2+Ag, 75 μg IRM2/mouse) or without (Ag alone) IRM2; DO.11.-transferred Balb/c mice were immunized with 100 μg OVA peptide (ISQAVHAAHAEINEAGR) per mouse, either with (IRM2+Ag, 75 μg IRM2/mouse) or without (Ag alone) IRM2.

On Day 3, the nasal associated lymphoid tissue was removed and the fold expansion of each cell population over PBS alone was determined. CD8⁺ OT-I cells were detected using SIINFEKL/K^{b} tetramers and CD4⁺ DO.11 cells were detected using a clonotypic antibody (Caltag Laboratories, Burlingame, Calif.) and analyzed using a FACSCaliber (Becton, Dickinson, San Jose, Calif.).

Results are shown in FIG. 3 as follows: CD8⁺ OT-I expansion is shown in FIG. 3A; CD4⁺ DO.11 expansion is shown in FIG. 3B. Intranasal immunization of an IRM/antigen combination induces expansion of both CD8⁺ T cells and CD4⁺ T cells to a greater extent than intranasal immunization with antigen alone.

### Example 3

Balb/c mice (Charles River Laboratories, Wilmington, Mass.) were treated with 50 μg of whole chicken ovalbumin (OVA) protein (Sigma-Aldrich, St. Louis, Mo.) with 50 μg of IRM4 in phosphate buffered saline (PBS) by various routes. Clean ovalbumin protein was prepared by washing the OVA with Bio-Beads (Bio-Rad Laboratories, Inc., Hercules, Calif., Cat#152-3920) to remove endotoxin, then resuspended in phosphate buffered saline (PBS). Mice were treated with OVA and IRM4 by sub-cutaneous (SC) injection, intra-venous (IV) injection, intra-muscular (IM) injection, intra-dermal (ID) injection, intranasal instillation (IN), intradermal OVA injection with topical administration of 10 μL of IRM4 cream directly over the OVA injection site (ID+Top.), or were left untreated (nothing).

On day 21, mice were sacrificed, lung and nasal lavages were performed by trachea administration of 1 mL of PBS and serum was obtained by cardiac puncture and centrifugation to remove cells. Serum was collected for analysis. Lavage samples were measured for OVA-specific IgA by ELISA. Serum samples were measured for OVA-specific IgG2a by ELISA.

The OVA specific antibody ELISAs were preformed by coating Costar EIR/RIA 96 well plates (Cat#3590, Corning, Inc., Corning, N.Y.) with 100 μL/well of a 20 μg/mL ovalbumin solution in PBS and incubated for one to two hours at 37° C. or overnight at 4° C. Plates were then washed one time with 0.5% Tween-20 in PBS solution (wash buffer). 200 μL/well of a 1% BSA in PBS solution were placed into the wells, and incubated for one to two hours at 37° C. or overnight at 4° C. Plates were then washed two times with wash buffer. Three-fold serial dilutions starting with undiluted lavage samples, or twenty-fold serial dilutions starting with a 1:10 dilution of serum samples were made across the plate in 0.2% BSA, 0.05% Tween-20 in PBS (dilution buffer) and incubated overnight at 4° C. Plates were then washed four times with wash buffer. 100 μL/well of a 1:2000 dilution of goat anti-mouse IgG2a (Southern Biotechnology Associates, Inc., Birmingham, Ala.) or goat anti-mouse IgA (Southern Biotechnology Associates, Inc.) in dilution buffer was placed into the wells and incubated at room temperature for one hour. Plates were then washed four times with wash buffer, filled with 100 μL/well of stabilized chromagen (Cat#SB02, Biosource International, Camarillo, Calif.), incubated for less than five minutes, and 50 μL/well of stop solution (Cat#SS02, Biosource International) were then added. Plates are read on a spectrophotometer at an OD of 490.

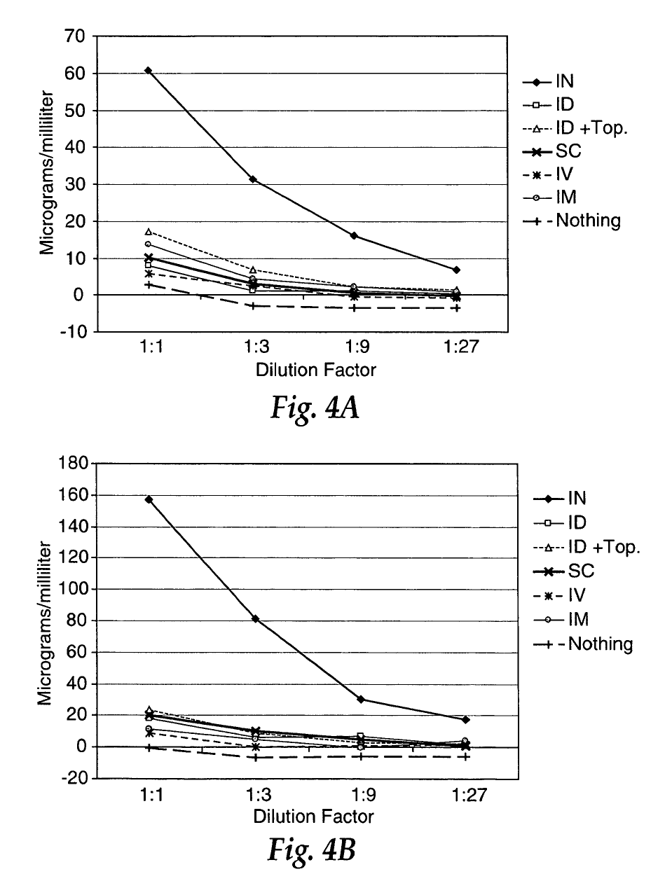

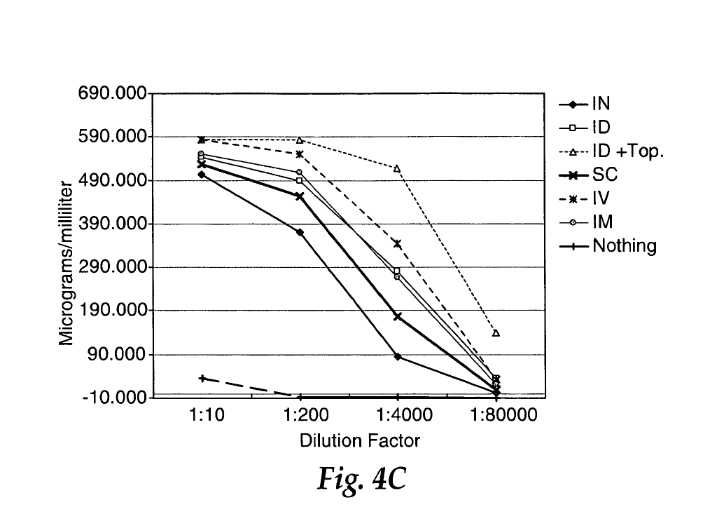

The results are shown in FIG. 4. Only intranasal administration of the IRM/antigen combination generated strong IgA responses in the lung (FIG. 4A) and nasal (FIG. 4B) mucosa. All routes of administration, including intranasal, generated strong IgG2a responses in the blood (FIG. 4C).

### Example 4

On Day 0 and Day 7 Balb/c mice (Charles Rivers Laboratories) were immunized intranasally with 35 μg of OVA alone or in combination with 14 μg of IRM3, IRM4, IRM5, IRM6, IRM7, IRM8, IRM9, IRM10, IRM11, or IRM12 in PBS. On Day 14, mice were sacrificed and lung lavage and serum collection was performed as described in Example 3. Lung lavage and serum samples were analyzed for OVA specific IgA and IgG2b (Southern Biotechnology Associates, Inc.), respectively, as described in Example 3.

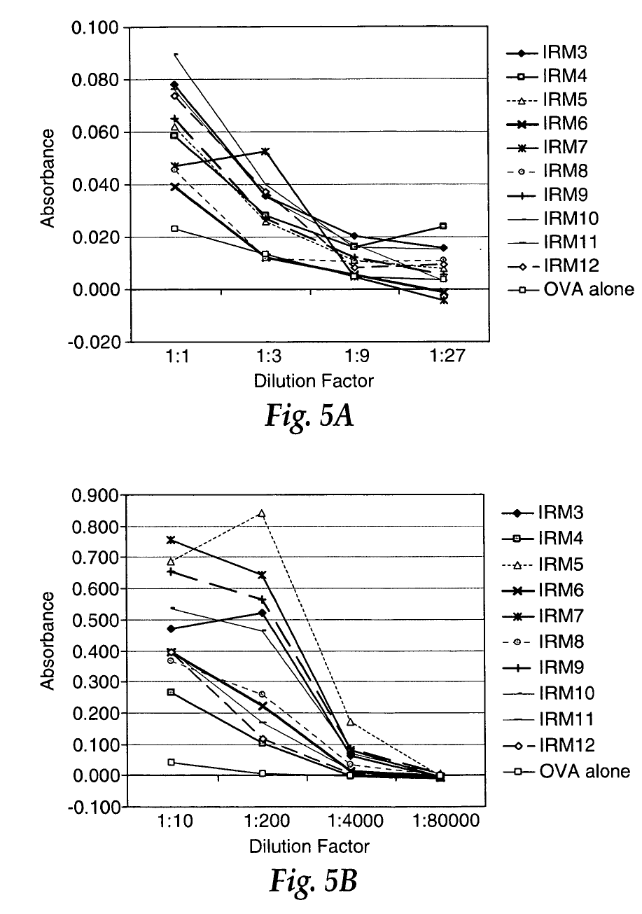

The results are shown in FIG. 5. IRM/antigen combinations of all IRM compounds tested provided greater IgA (FIG. 5A) and IgG2b (FIG. 5B) responses than antigen alone.

### Example 5

Lymphocytes from lymph nodes of GFP+/OT-I+C57BL6 mice were adoptively transferred into C57BL6 mice. One day after adoptive transfer, the mice were immunized nasally with 35 μg of ovalbumin alone or in combination with 14 μg of IRM3, IRM4, IRM5, IRM6, IRM7, IRM8, IRM9, IRM10, IRM11, or IRM12 in citrate buffered saline (CBS). Four days later, mice were sacrificed and draining lymph nodes (DLN) and spleens were removed. The total number of DLN lymphocytes and splenocytes were determined by using a Guava PCA 96 (Guava Technologies, Inc., Hayward, Calif.). DLN lymphocytes and splenocytes were stained with propidium iodine (PI) and mouse anti-CD8 antibody (BD Pharmingen, San Diego, Calif.) and the percentage of OT-I⁺/GFP⁺ lymphocytes was determined by flow cytometry gating on PI⁻CD8⁺/GFP⁺ lymphocytes. The total number of OT-I⁺/GFP⁺ lymphocytes was determined by multiplying the total number of splenocytes by the percent PI⁻OT-I⁺/GFP⁺ lymphocytes.

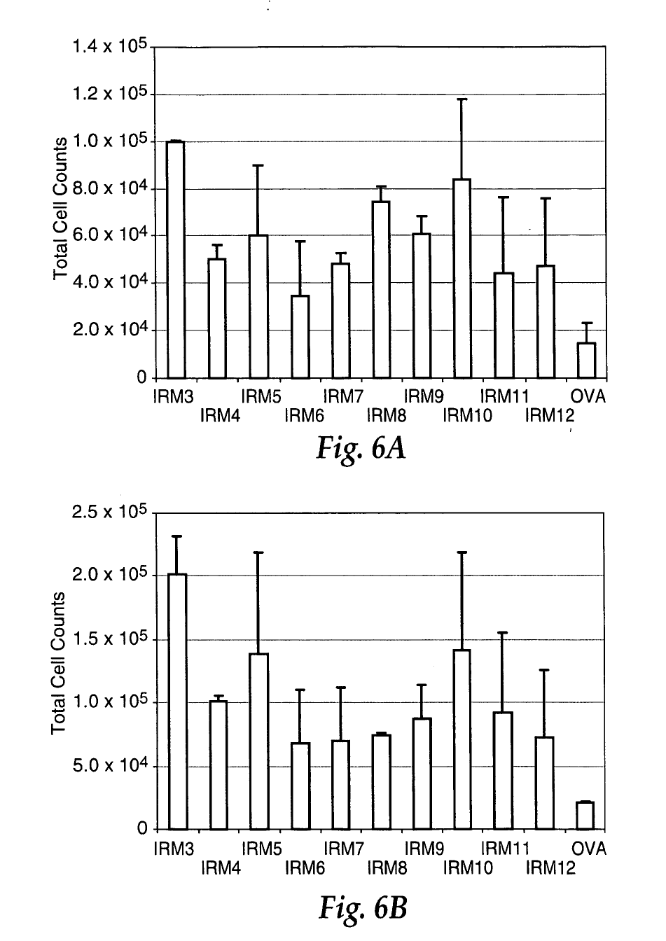

The results are shown in FIG. 6. Intranasal administration of IRM/antigen combinations employing many different IRM compounds provided greater number of antigen-specific T cells in the DLN (FIGS. 6A) and the spleen (FIGS. 6B) than administration of antigen alone.

### Example 6

Lymphocytes from OT-I mice (The Jackson Laboratories, Bar Harbor, Me.) were adoptively transferred into C57BL/6 (Charles River Laboratories, Wilmington, Mass.) mice. Ovalbumin was washed as described in Example 3. One day after the adoptive transfer, mice were immunized with PBS alone intranasally or 50 μg of ovalbumin and 50 μg of IRM4 in PBS intranasally (IN), intravenously (IV), or subcutaneously (SC). Five months later, mice were either immunized again in the same manner they had been immunized previously, or were not re-immunized. Mice were sacrificed four days after the five-month immunization and the draining lymph nodes (DLN) and nasal associated lymphoid tissue (NALT) were collected.

The total number of DLN lymphocytes and NALT lymphocytes were determined by using a Guava PCA 96 (Guava Technologies, Inc., Hayward, Calif.). DLN lymphocytes and NALT lymphocytes were stained with propidium iodine (PI) and mouse anti-CD8 antibody (BD Pharmingen, San Diego, Calif.) and the percent of OT-I⁺/GFP⁺ lymphocytes was determined by flow cytometry gating on PI⁻CD8⁺/GFP⁺ lymphocytes. The total number of OT-I⁺/GFP⁺ lymphocytes was determined by multiplying the total number of DLN lymphocytes or NALT lymphocytes by the percent DLN or NALT PI⁻OT-I⁺/GFP⁺ lymphocytes.

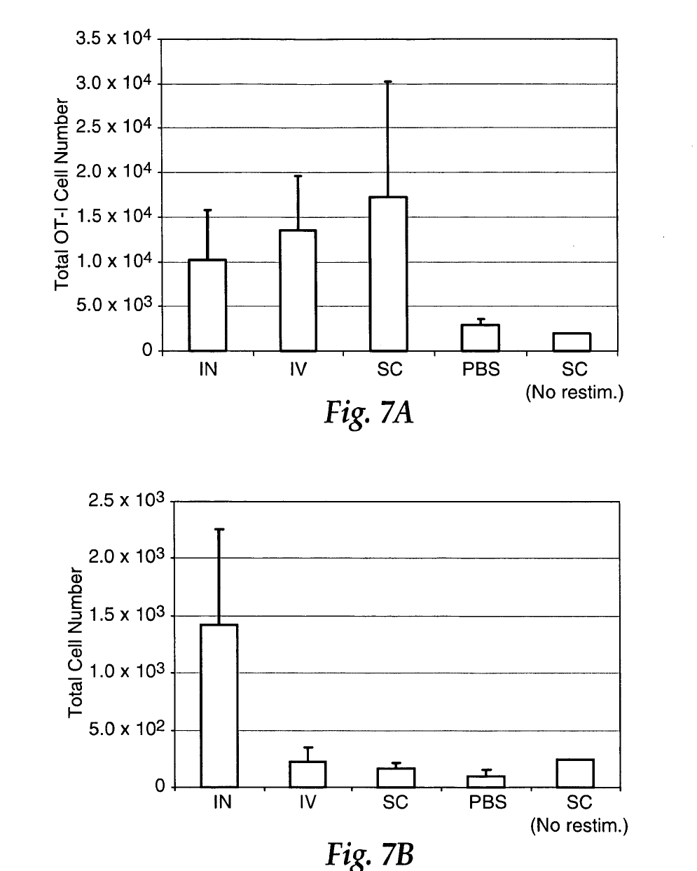

The results are shown in FIG. 7 as follows: DLN in FIG. 7A; NALT in FIG. 7B. All routes of immunization, including intranasal, caused an increase in OT-I cell number in the DLN upon re-immunization. Furthermore, intranasal immunization caused an increase in OT-I cell number in the NALT upon re-immunization.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A pharmaceutical combination comprising:
an IRM compound formulated for mucosal administration; and
an antigen formulated for mucosal administration.

2. The pharmaceutical combination of claim 1 comprising a single formulation that comprises the IRM compound and the antigen.

3. The pharmaceutical combination of claim 1 comprising:
a first formulation that comprises the IRM compound; and
a second formulation that comprises the antigen.

4. A method of immunizing a subject comprising:
administering an antigen to a mucosal surface of the subject in an amount effective, in combination with an IRM compound, to generate an immune response against the antigen; and
administering an IRM compound to a mucosal surface of the subject in an amount effective, in combination with the antigen, to generate an immune response against the antigen.

5. The method of claim 4 wherein the antigen and IRM are administered in one formulation.

6. The method of claim 4 wherein the antigen is administered in a first formulation and the IRM compound is administered in a second formulation.

7. The method of claim 6 wherein the antigen and the IRM compound are administered at different sites.

8. The method of claim 7 wherein at least one site comprises nasal mucosa.

9. The method of claim 7 wherein at least one site comprises oral mucosa.

10. The method of claim 7 wherein at least one site comprises gastro-intestinal mucosa.

11. The method of claim 7 wherein at least one site comprises urogenital mucosa.

12. The method of claim 7 wherein the different sites are different mucosal surfaces.

13. The method of claim 6 wherein the IRM compound is administered before the antigen is administered.

14. The method of claim 6 wherein the IRM compound is administered after the antigen is administered.

15. The method of claim 4 wherein the antigen comprises a protein, a peptide, a live or inactivated bacterium, a live or inactivated virus, or any combination thereof.

16. The method of claim 4 wherein the IRM compound comprises a 2-aminopyridine fused to a five membered nitrogen-containing heterocyclic ring.

17. The method of claim 4 further comprising at least one additional administration of the antigen.

18. The method of claim 4 further comprising at least one additional administration of an IRM compound.

19. The method of claim 18 wherein the IRM compound of the first administration of IRM compound is different than the IRM compound of the second administration of IRM compound.

20. The method of claim 4 wherein the immune response against the antigen comprises secretion of IgA.

21. The method of claim 4 wherein the immune response against the antigen comprises increasing the number or percentage of antigen-specific T cells in a mucosal tissue.


SEQUENCE LISTING (2 sequences)

SEQ ID NO: 1 - protein, 8 aa, Artificial
  Ovalbumin partial peptide
SIINFEKL

SEQ ID NO: 2 - protein, 17 aa, Artificial
  Ovalbumin partial peptide
ISQAVHAAHAEINEAGR